(19) United States
(12) Patent Application Publication
(10) Pub. No.: US 2009/0259320 A1
(43) Pub. Date: Oct. 15, 2009

(54) Generator for prosthesis and orthosis

(76) Inventor: Jan Andrysek, Toronto (CA)

Correspondence Address: MILLER THOMSON LLP, 100 STONE ROAD WEST, SUITE 301, GUELPH, ON, N1G-5L3, CA

(21) Appl. No.: 12/310,880

(22) PCT Filed: Sep. 17, 2007
(86) PCT No.: PCT/CA2007/001624

§ 371 Date: Mar. 11, 2009

Related U.S. Application Data
(60) Provisional application No. 60/844,669, filed on Sep. 15, 2006.

(51) Int. Cl. A61F 2/70

(52) U.S. Cl. 623/24

(57) ABSTRACT

A means for using body energy to generate electrical current to power the electronics, sensors, actuators and other electronic components in microprocessor-controlled prosthetic or orthotic joints. Furthermore, a means for using the generator to provide swing-phase damping is disclosed whereby the damping level can be controlled and adjusted electronically.

### FIELD OF INVENTION

This invention relates generally to a power generating method and system that uses body energy from gait to operate electronic prosthetic or orthotic devices.

### BACKGROUND OF INVENTION

Artificial joints generally require mechanisms to control their movement. For example an artificial knee joint or prosthetic joint will be prescribed for a person with a through-knee (TK) or an above-knee (AK) amputation. i.e. a person without a knee joint, shank or foot. The ability for the knee to bend or articulate during sitting, kneeling or ambulating is desirable. It is also desirable to have the ability to control the leg during the swing and stance-phases of gait when the person is walking or running.

The “swing-phase control” refers to the control of the joint's movement or articulation during the swing-phase of the gait cycle to make the gait more efficient and more natural looking. Traditionally fluid-based pneumatic or hydraulic dampers are used in prosthetics to help control the swing-phase. With these devices, the control is initially tuned to suit the walking patterns of the amputee. However, if the amputee significantly alters his/her walking pattern, the knees will require further adjustment on the part of the prosthetist. Furthermore, fluid-based dampers tend to be complex and susceptible to leaks.

Microprocessor-controlled, electronically or computer controlled prostheses all utilize feedback control to better adapt to changes in the amputee's gait, and continually adjust the level of damping. This facilitates a more natural and efficient gait for individuals with above-knee amputations. Several devices are currently on the market. The Blatchford Endolite Intelligent prostheses use a pneumatic damper that is continually adjusted to provide optimal damping for swing-phase control. The C-leg prosthesis uses a microprocessor-controlled hydraulic damper for both swing-phase and stance-phase control. The Rheo knee uses a similar approach, but instead of hydraulic fluid, it uses magnetorheological fluid. One drawback of these technologies is that the user must charge the on-board batteries on a daily basis. A second drawback is that the systems tend to be heavy and bulky, in part due to the battery packs.

Historically, the application of pneumatic and hydraulic dampers for prosthetic swing-phase control has been successful because controlling motions at the knee joint can be done very well by applying torque passively. This means that energy is dissipated in pneumatic and hydraulic dampers predominantly in the form of heat. However, instead of dissipating the energy, it is possible to convert and store the energy, so that it may power the electronics in microprocessor-controlled prostheses. This eliminates the need for large and heavy battery packs, and also the inconvenience associated with daily charging of batteries.

The use of physiological energy to charge batteries and/or power electronic devices has mainly been applied to able-bodied gait, and in the form of generators located in the shoe. These are described in several patents namely U.S. Pat. Nos. 6,182,378 and 6,255,799, JP Patent Nos. 2001327197 and JP2006014572, and CN Patent Nos. 1202340, CN 1541582 (2004), and CN1707904 (2005).)

### SUMMARY OF INVENTION

The object of one aspect of the present invention is to provide a means for using body energy from the gait to provide power to electronic prosthetic or orthotic systems. An alternative objective is to provide a means for using body energy from the gait to provide swing-phase damping. Another objective it is to provide concomitantly power to electronic prosthetic systems and swing-phase damping

In accordance with one aspect of the present invention there is provided an artificial knee joint with a means to generate electrical current from the motions (activities) of the prosthesis or orthosis during walking, jogging or running For convenience, an electromechanical generator such as a DC motor is applied to a knee joint, so that relative motions between the tipper (thigh) and lower (shank) portions of the knee joint will drive the motor rotor. A transmission means, such as a gear assembly, is used to amplify the motions at the knee joint. in order to increase the rotor speed, and facilitate the generation of adequate levels of current. This electrical current may be used to recharge onboard batteries, or directly to power the electronics of a microprocessor-based prosthesis, by way of example only and therefore eliminating the need for a battery pack.

The other aspect of the invention relates to the use of the generator namely a geared motor to provide damping. By decreasing the electrical resistance between the motor terminals, generated current is allowed to flow back into the geared motor. This increases the resistance in the motor, in effect causing the motor through the transmission to act as a damper.

In one application, a smaller geared motor can be used in a microprocessor-based prosthesis or orthosis for mainly current generation, to supply power to the electronics and/or to keep onboard batteries charged. This would be applicable to prosthetic technologies that use electronics, such as the aforementioned commercially-available microprocessor-based knee joints.

In another application. a larger geared motor can be used to provide damping, much like a pneumatic or hydraulic damper. This may be applicable to conventional prosthetic technologies that do not use electronics and as a substitute for more costly and higher-maintenance hydraulic or pneumatic dampers. Preferably a variable resistor would be used to tune the amount of current that is redirected back into the motor, and ultimately the damping level, much like adjusting the valve on a hydraulic or pneumatic damper. Finally, a larger geared motor may be used to generate power to supply the electronics in a microprocessor controlled knee joint, and in addition provide a means for damping.

The device could be based on an electromagnetic, piezoelectric or other type of means of electrical current generation. It may by used to power any type of prosthesis that uses electronics, and hence requires a supply of power. The device may be applied at any prosthetic or orthotic joint for example at the knee, ankle, elbow, hip or shoulder, and may generate electrical current during the swing-phase of gait, stance-phase of gait, or both phases. For example tie passive moments at the prosthetic ankle during stance can be used to generate electrical current for a microprocessor-based prosthetic knee joint In a single prosthesis.

### BRIEF DESCRIPTION OF DRAWINGS

A detailed description of the preferred embodiments is provided herein below by way of example only with reference to the following drawings, in which:

FIG. 1 illustrates an artificial knee joint with a means for current generation

FIG. 2 illustrates the circuit used to rectify generated current, and control the flow of current to the batteries for charging and swing-phase damping.

FIG. 3 shows data that characterize the damping of a geared motor.

FIG. 4 shows data that characterize the current generation of a geared motor.

FIG. 5 shows data of current generated during walking for an amputee.

FIG. 6 shows the application of the geared motor for use in a microprocessor-based knee joint.

FIG. 7 shows the application of the geared motor for used in a knee joint with polycentric stance-phase control.

FIG. 8 shows data of damping torques produced by the geared motor during walking

In the drawings, preferred embodiments of the invention arc illustrated by way of example. It is to be expressly understood that the description and drawings are only for the purpose of illustration and as an aid to understanding, and are not intended as a definition of the limits of the invention

### DESCRIPTION OF THE PREFERRED EMBODIMENT

The following description relates to the preferred embodiments of the present invention for a generator in a prosthetic or orthotic joint. In accordance with a preferred embodiment of the present invention there is provided a generator for a prosthesis having a means for generating electrical current using body energy transmitted to the prosthesis. Body energy may be further defined as energy emanating from activities from the body such as mechanical, vibrational, psychological, thermal, ultrasonic (sound waves via muscles), or biochemical body energy, current by way of example only, that is then transmitted to the prosthesis and converted into an electrical current. The mode of transmission of the body energy to the prosthesis may vary depending an the type of body energy being generated. For example, mechanical body energy may be generated and transmitted by the movement of the body or through a force being exerted within the body.

The means for generating electrical current using body energy transmitted to the prosthesis may be further defined as a means for converting body mechanical energy adapted to engage the prosthesis to generate an electrical current. The means for generating electrical current using body energy transmitted to the prosthesis further includes a transmission means adapted to engage the prosthesis to generate the electrical current by the means for converting body mechanical energy.

The means for generating electrical current using body energy transmitted to the prosthesis may also be defined as an electromechanical generator and the means for converting body mechanical energy may be defined as an electromechanical motor. A brushed direct current (DC) motor may be used. The transmission means may be a gear assembly that allows for the amplification of the body energy transmitted to the prosthesis. The electromechanical generator can further provide damping to control the movement of the prosthesis. Specifically a simple electronic circuit can be used to vary how much generated current is allowed to flow back into the geared motor. For example, by connecting the negative and positive terminals of a brushed direct current motor, in effect achieving a short circuit, the maximum level of damping is achieved. Therefore, as the motor rotor is mechanically driven this motion is resisted by a high damping torque which increases as the driving speed of the rotor increases. Conversely, minimal damping torque is achieved by disconnecting the terminals of the motor, so that an open circuit is achieved. When the motor is mechanical driven in this open circuit setup, there is minimal damping torque resisting the motion of the motor. Finally the current generated by the means for generating electrical current using body energy transmitted to the prosthesis may also be used to power electronics in a microprocessor-controlled prosthesis.

The means for generating electrical current using body energy transmitted to the prosthesis may also be defined as a piezoelectric generator or an electromagnetic generator by way of example only. The current generated by the means for generating electrical current using body energy transmitted to the prosthesis may be used to facilitate the operation of the prosthesis. For example the current may be used to recharge onboard batteries or be used to power electronics in a controlled prosthesis.

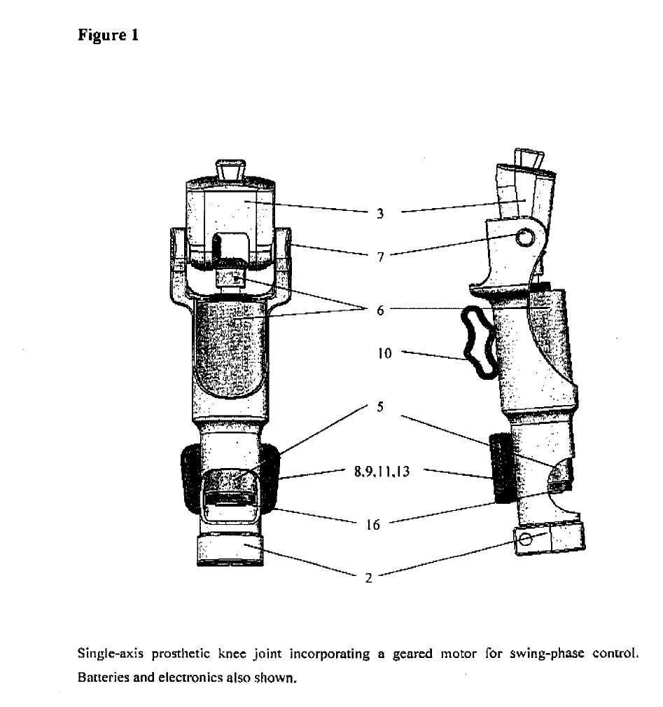

FIGS. 1 to 8 illustrate the instant invention in operation. FIG. 1 illustrates a single-axis prosthetic knee joint [20] with a geared motor for swing-phase control. Generator [5] converts human mechanical energy that is normally dissipated during the swing-phase to electrical energy so that it may be used to power an onboard microprocessor. sensors, actuators and associated electronics used to control the damping in a prosthetic or orthotic knee joint. The proposed embodiment utilizes an electromechanical generator [5], but other means for generating electricity could be applied such as electromagnetic or piezoelectric generator. The electromechanical generator [5] is essentially a DC brushed motor that is mechanically driven to generate electricity. The speed with which the motor is driven is amplified using gears [6]. so that slow speeds at the output shaft [7] of the gears result in a fast rotation of the motor rotor. This is necessary because the motions at the knee joint during walking are relatively slow, while the rotor speed that is needed to generate electrical current is relatively high.

In one embodiment, the geared motor is located in the shank portion [2] of the prosthetic knee joint and the output shaft is linked by gears [6] to the thigh portion [3] of the knee prostheses (FIG. 1). This allows the motions at the knee (knee flexion and extension) to drive the motor, and in effect generate electrical current. The faster the motor is driven the greater the potential (voltage) developed across the motor terminals. Placing batteries [10] at the terminals allows current to flow and charge the batteries. Alternatively current can be stored using capacitors or other electronic means. It should be evident that current will not flow and battery charging will not occur until the potential at the motor terminals exceeds that of the batteries. The battery potential will be selected to adequately power the microprocessor and onboard electronics [13]. For example five 1.2 V Nickel metal hydride cells would be adequate to power a 5 V circuit. Therefore, charging of batteries will not occur for motor speeds that are below a certain threshold

Due to the oscillatory nature of the knee motion during walking, resulting from knee flexion and extension cycles. the current generated is alternating. A rectifying circuit is needed to convert it to direct current so that it can be used by the electronics or to charge the batteries. As the amputee walks with this device, pulses of current are generated, corresponding to peak knee flexion and knee extension angular velocities.

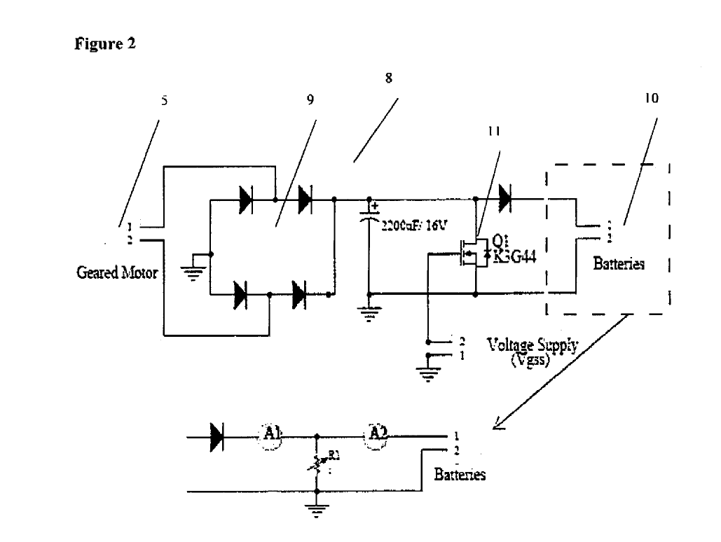

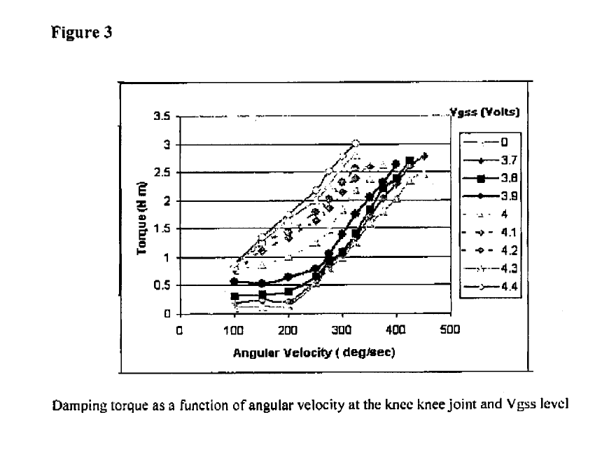

FIG. 2 illustrates the basic circuit [8] described above that includes the rectifying circuit [9] and batteries [10]. In addition the circuit illustrates a means [11] for controlling how much of the generated current is directed to the batteries, with the remainder being directed back into the motor Redirecting generated current back to the motor causes the motor to resist motion, and in effect provide damping. If all of the generated current is redirected back into the motor, for example by connecting the motor terminals together, the motor will produce passive torques when driven that are essentially proportional to the driving speed. FIG. 3 illustrates this relationship, as based on experimental data. This speed dependent passive damping is a desired characteristic in prosthetic swing-phase control and characteristic of traditional hydraulic and pneumatic-based systems. But, further, by controlling the amount of current that is redirected back to the motor, for example using a MOSFET [11] as shown in FIG. 2, the amount of damping can be adjusted, essentially on a real tine basis. This gives means for using the geared motor for swing-phase control that can adapt to an amputee's gait using a microprocessor-based feedback control system.

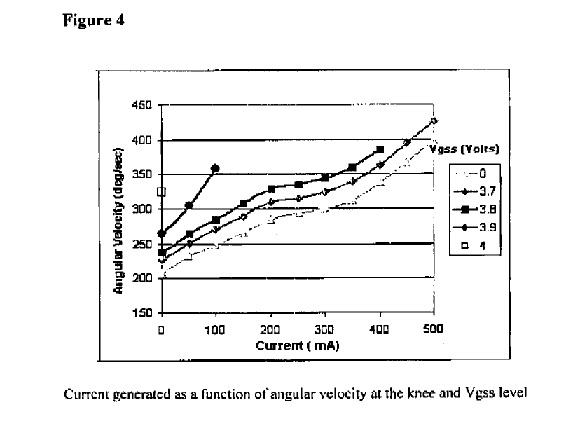

FIG. 3, illustrates the damping torques as a function of angular velocity at the knee and the level of current flow back into the motor, as regulated by the MOSFET voltage V_{GSS}. FIG. 4 shows the current that is generated for the various levels of damping (V_{GSS}). As more and more current is directed to the motor to increase damping (i.e. higher V_{GSS}). less current is available for charging batteries. Therefore, the feasibility of the system is analyzed in three respects, namely does the system produce an adequate charging current, does the system provide adequate swing-phase damping, and does the system produce adequate charging and adequate swing-phase control, concurrently.

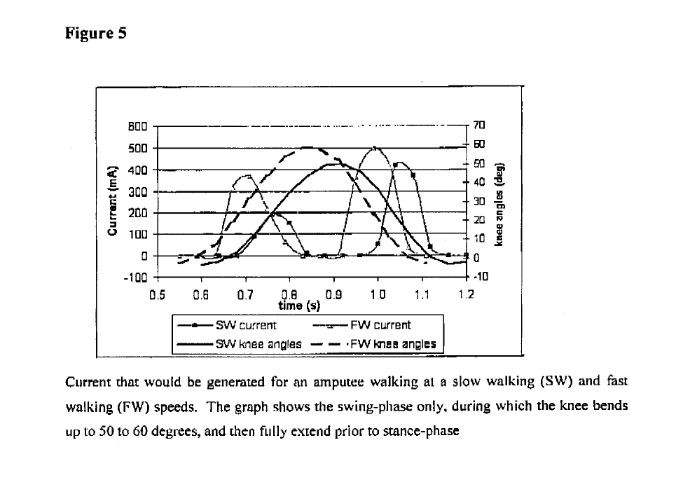

The charging characteristics of the geared motor as shown in FIG. 4 were applied to the walking data, specifically the knee angular velocities of an amputee (FIG. 5). The data are based on the circuit design in FIG. 2 with five 1.2 V battery cells in series [10]) for a total of 6 V. Charging occurs in pulses that correspond Lo instances of high knee angular velocity. These pulses of charging current are generally large in magnitude, but short in duration. Because charging of batteries occurs for knee angular velocities above a threshold, in this case about 200 deg/s (FIG. 4), the data in FIG. 5 shows parts of the phase when no charging current is produced.

In order to maximize charging current. a number of design parameters can be affected. The gear ratio should be selected to maximize rotor speed. The gear ratio should however not be as high so as to exceed the maximum rated speed for the motor, or prevent back driving of the motor. Gear ratios between 50 and 500 may be optimal. The specifications of the motor can also influence the power generated. A larger motor will generally produce more current. A motor with a higher velocity constant (i.e. a motor that runs at a higher voltage) will generate a higher potential across the terminals, at lower rotor speeds when mechanically driven. This will decrease the threshold for charging, for example, from 200 deg/s to 100 deg/s. The threshold speed can also be decreased by using a lower voltage battery pack, for example four 1.2 V cells (total of 4.8 V) that might adequately power a 3.7 V microprocessor circuit. The reduction in threshold can also be accomplished by charging batteries in parallel, so that for example in the design presented here the threshold would be based on 1.2 V. A reduced threshold allows longer pulses of charging current, and a smoother charging profile.

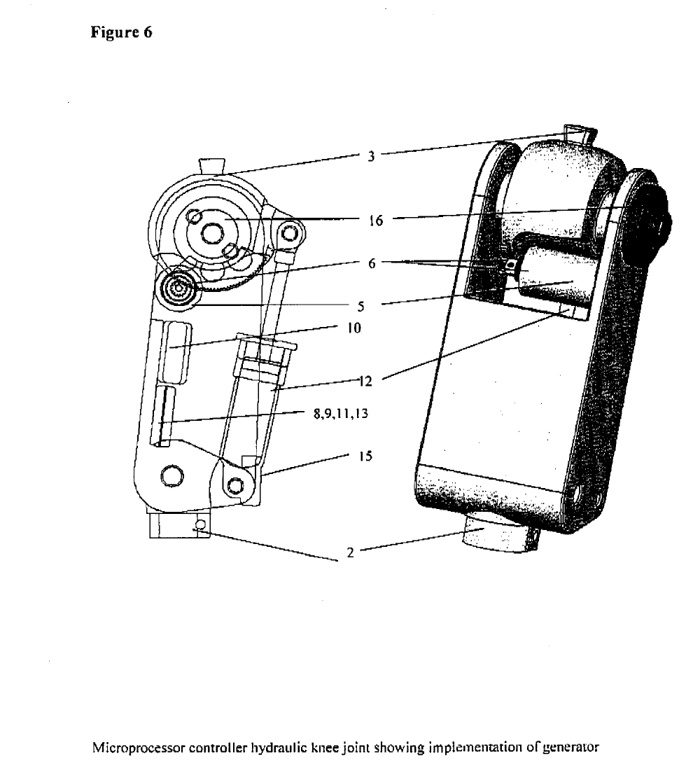

The generator may be designed into existing systems. for example a microprocessor controlled hydraulic based swing-phase/stance-phase controller such as the C-leg. An example of this is presented in FIG. 6. FIG. 6 shows a single-axis prosthetic knee joint with a hydraulic damper [12] that provides both, swing-phase and stance-phase control. Sensors provide information to a processor [13] about the performance of the prostheses, and appropriate signals to actuators that alter the level of damping. The generator works predominantly during the swing-phase, to continually replenish a small battery pack [10] that supplies power to the microprocessor [13], actuators [15] that regulate the fluid flow in the hydraulic damper, and sensors [16] that detect, for example the instantaneous position in the knee joint.

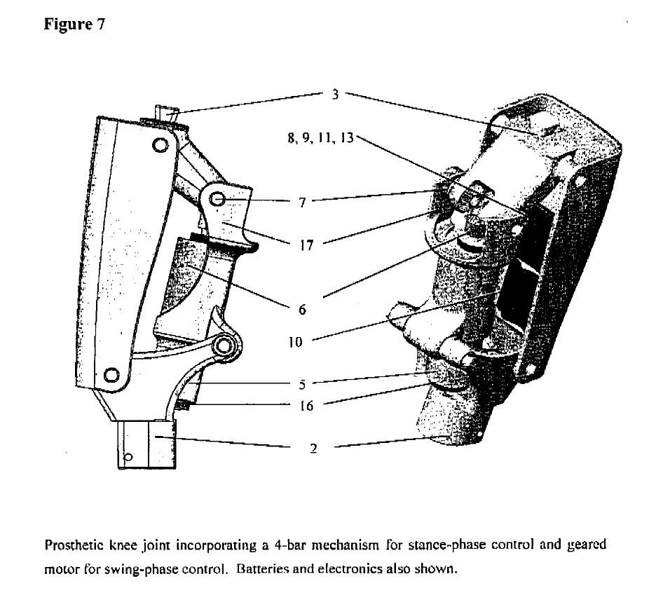

The geared motor can also be utilized for adaptable swing-phase damping, as mentioned above. the mechanisms would generally be applied as in FIG. 1, or for better stance-phase control a four-bar linkage mechanism. FIG. 7 shows the application of the geared motor to a polycentric knee joint. The geared motor transmits by gears [6] the relative motion between two of the linkages. in this case the top (thigh) [3] and rear linkage [17]. The amount of knee flexion is sensed [16] and used as feedback in an electronic circuit to regulate the V_{GSS} voltage, and in effect the swing-phase damping. In this case, current that is not used for damping, charges the batteries

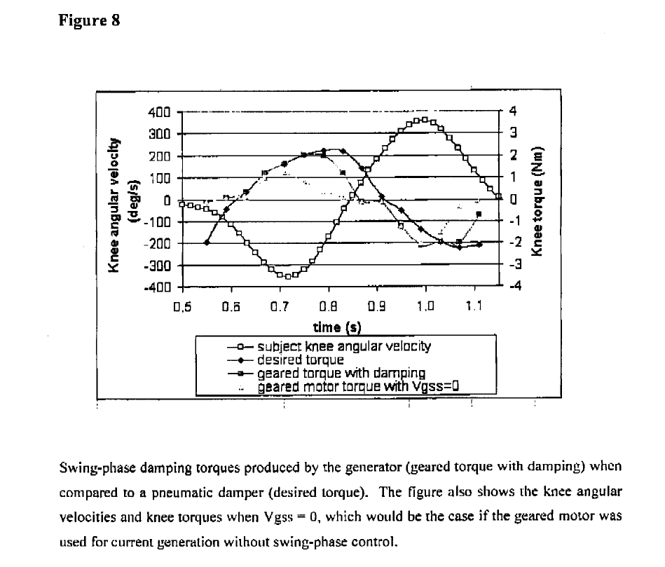

FIG. 8 shows the torques produced by the geared motor during walking. Swing-phase damping can be tuned to closely match the damping characteristics of a conventional hydraulic or pneumatic damper. Alternatively, the damping characteristics of the geared motor can be adjusted on the fly, in order to provide better swing-phase control than a conventional hydraulic or pneumatic damper. This is achieved via closed-loop microprocessor-based control.

Other variations and modifications of the invention are possible. All such modifications or variations are believed to be within the sphere and scope of the invention as defined by the claims appended hereto.

1. A generator for a prosthesis comprising a means for generating electrical current using body energy transmitted to the prosthesis.

2. A generator for a prosthesis as claimed in claim 1 wherein the means for generating electrical current using body energy transmitted to the prosthesis is a means for converting body mechanical energy adapted to engage the prosthesis to generate an electrical current.

3. A generator for a prosthesis as claimed in claim 2 further comprising a transmission means adapted to engage the prosthesis to generate the electrical current by the means for converting body mechanical energy.

4. A generator for prosthesis as claimed in claim 3 wherein the means for generating electrical current using body energy transmitted to the prosthesis is an electromechanical generator.

5. A generator for prosthesis as claimed in claim 4 wherein the means for converting body mechanical energy is an electromechanical motor.

6. A generator for prosthesis as claimed in claim 5 wherein the transmission means is a gear assembly for amplification of the body energy transmitted to the prosthesis.

7. A generator for prosthesis as claimed in claim 6 wherein the electromechanical generator further provides damping to control the movement of the prosthesis.

8. A generator for prosthesis as claimed in claim 7 wherein the current generated by the means for generating electrical current using body energy transmitted to the prosthesis is used to power electronics in a microprocessor controlled prosthesis.

9. A generator for prosthesis as claimed in claim 1 wherein the means for generating electrical current using body energy transmitted to the prosthesis is a piezoelectric generator.

10. A generator for prosthesis as claimed in claim 1 wherein the means for generating electrical current using body energy transmitted to the prosthesis is an electromagnetic generator,

11. A generator for prosthesis as claimed in claim 1 wherein the current generated by the means for generating electrical current using body energy transmitted to the prosthesis is used to facilitate the operation of the prosthesis.

12. A generator For prosthesis as claimed in claim 1 wherein the current generated by the means for generating electrical current using body energy transmitted to the prosthesis is used to power electronics in microprocessor controlled prosthesis.

13. A generator for prosthesis as claimed in claim 1 wherein the prosthesis is a knee, ankle, shoulder, hip or elbow joint.

14. A generator for prosthesis as claimed in claim 1 wherein the current is generated during the swing phase of gait, the stance-phase of gait or during both phases.

15. A generator for an orthosis comprising a means for generating electrical current using body energy transmitted to the orthosis.

16. A generator for an orthosis as claimed in claim 15 wherein the means for generating electrical current using body energy transmitted to the orthosis is a means for converting body mechanical energy adapted to engage the orthosis to generate an electrical current.

17. A generator for an orthosis as claimed in claim 16 further comprising a transmission means adapted to engage the orthosis to generate the electrical current by the means for converting body mechanical energy.

18. A generator for an orthosis as claimed in claim 17 wherein the means for generating electrical current using body energy transmitted to the orthosis is an electromechanical generator.

19. A generator for an orthosis as claimed in claim 18 wherein a means For converting body mechanical energy is an electromechanical motor.

20. A generator for an orthosis as claimed in claim 19 wherein the transmission means is a gear assembly for amplification of the body energy transmitted to the orthosis.

21. A generator for an orthosis as claimed in claim 20 wherein the electromechanical generator further provides damping to control the movement of the orthosis.

22. A generator for an orthosis as claimed in claim 21 wherein the current generated by the means for generating electrical current using body energy transmitted to the orthosis is used to power electronics in microprocessor controlled orthosis.

23. A generator for an orthosis as claimed in claim 15 wherein the means for generating electrical current using body energy transmitted to the orthosis is a piezoelectric generator.

24. A generator tor an orthosis as claimed in claim 15 wherein the means for generating electrical current using body energy transmitted to the orthosis is an electromagnetic generator.

25. A generator For an orthosis as claimed in claim 15 wherein the current generated by the means for generating electrical current using body energy transmitted to the orthosis is used to facilitate the operation of the orthosis.

26. A generator for an orthosis as claimed in claim 15 wherein the current generated by the means for generating electrical current using body energy transmitted to the orthosis is used to power electronics in microprocessor controlled orthosis.

27. A generator for an orthosis as claimed in claim 15 wherein the orthosis is a knee, ankle, shoulder, hip or elbow joint.

28. A generator for an orthosis as claimed in claim 15 wherein the current is generated during the swing phase of the gait, the stance-phase of the gait or during both phases.
